(19) United States
(12) Patent Application Publication
(10) Pub. No.: US 2024/0042383 A1
(43) Pub. Date: Feb. 8, 2024

(54) METHOD FOR AIR PURIFICATION AND SIMULTANEOUS PRODUCTION OF O2 BY MEANS OF ALGAL CULTURE

(71) Applicant: ALOS S.R.L., Egna (Bolzano) (IT)

(72) Inventors: Emiliano Gentilini, Terlano (Bolzano) (IT); Francesco Guzzonato, Trento (IT); Andrea Dal Negro, Bolzano (IT); Claudio Gobber, Prade - Canal San Bovo (Trento) (IT)

(21) Appl. No.: 18/039,824

(22) PCT Filed: Dec. 2, 2021
(86) PCT No.: PCT/IB2021/061236

§ 371 Date: Jun. 1, 2023

(30) Foreign Application Priority Data
Dec. 4, 2020 (IT) 102020000029951
Dec. 4, 2020 (IT) 102020000029957

(51) Int. Cl. B01D 53/84; B01D 53/78; B01D 53/00; B01D 53/62; B01D 53/96; C12N 1/12

(52) U.S. Cl. CPC B01D 53/84; B01D 53/78; B01D 53/007; B01D 53/62; B01D 53/96; C12N 1/12; B01D 2251/95; B01D 2257/504; B01D 2258/06; B01D 2259/4508; B01D 2259/802

(57) ABSTRACT

The present invention relates to the field of air purification of indoor and/or domestic environments and has as its object a method for air purification and simultaneous photo-conversion of carbon dioxide to oxygen by exploiting a photosynthesis process of an algal culture comprised within an aqueous medium. The method according to the present invention therefore allows to purify the air of an indoor environment by abatement/solubilization of pollutants in the aqueous medium and reduction of CO₂ contraction by photo-conversion into O₂. The method according to the present invention further comprises the steps of illuminating and aerating said aqueous medium as well as a step of periodically maintaining said algal culture in a state of constant growth in order to optimize the photosynthesis of the algal culture.

### FIELD OF THE INVENTION

The present invention relates to the field of air purification of indoor and/or domestic environments and has as its object a method for air purification and simultaneous photo-conversion of carbon dioxide to oxygen by exploiting a photosynthesis process of an algal culture.

### PRIOR ART

Today, living conditions within an indoor environment are increasingly the subject of research and technological development, especially in the light of an increasingly widespread use of working methods such as smart (or agile) working, which allows workers to fulfil their work tasks, for example through remote access from a company computer, without the need to go to the office. In essence, the domestic environment increasingly assumes the connotation of a real work station. Therefore, the improvement of living conditions within such environments has become an essential parameter for ensuring the worker's health. In particular, the quality of the air breathed is one of the fundamental parameters for determining the living conditions of an environment. In fact, it should be underlined that air quality is a recurring theme in studies on the quality of life of the population. Several studies have shown that the level of pollutants which accumulate in an enclosed space can be equal to, or even greater than, that present in the outdoor environment. This problem takes on particularly obvious connotations not only in particularly industrialized urban conglomerates but also in cities where the multitude of vehicles circulating leads to a high amount of pollution: the well-known smog. It is estimated that more than twenty thousand people die from urban pollution every day. In fact, human activities release toxic substances into the air, responsible for a total 12.5% of all deaths in the world.

Therefore, the increasing attention to the quality of living conditions, and the consequent interest in improving the liveability of both domestic and work environments, have led to the development of increasingly advanced methods to try to remove, or at least render harmless, the substances harmful to human health which are present in the air.

Currently in the domestic air purification sector, methods and devices are used which are based on physical filtering technologies, electronic filtering, or gas phase filtering. Examples of these technologies are HEPA filters, air ionizers and activated carbon filters, respectively. In addition, other household air purification strategies use the photocatalytic properties of titanium dioxide. In particular, in the presence of UV radiation, titanium dioxide is able to photocatalytically destroy the pollutants present in the air, degrading them to H₂O and CO₂.

Other solutions are instead based on the so-called “water revitalizers”, i.e., tanks filled with water which exploit the generation of a slight wave motion in order to favour the water-air exchange surface and thus retain the pollutants present in the latter. Lastly, another technological solution mainly used at industrial level is based on the use of an algal culture in a continuous system, in large water tanks which exploit solar radiation in order to purify the air from pollutants.

However, one of the most common drawbacks of such methods of the known art is the poor adaptability to the different environments in which they can be used.

In fact, the air filtration technologies mentioned above are based on devices which are specifically sized according to the size of the environment within which they will be arranged.

This feature severely limits the adaptability and operational flexibility of the air purification method, since the use of an under-sized system results in poor purification efficiency and, conversely, the use of an over-sized system leads to unjustified energy waste.

Disadvantageously, moreover, the effectiveness of the elements with photocatalytic properties used for the air purification rapidly degrades, leading to recurrent maintenance costs.

Furthermore, the purification methods currently available in the current state of the art have onerous costs related to the operation thereof and often use devices with inherently large dimensions (due for example to the presence of voluminous fans for air intake or large culture tanks) which limit the use thereof in smaller domestic environments. In fact, such devices generally cause an at least partial obstruction of the light sources present within the environment itself, limiting the diffusion of light. Furthermore, another disadvantage mainly related to the systems based on the use of an algal culture concerns the costs and operations related to keeping alive and optimizing the photosynthesis conditions of the algal culture itself.

Therefore, the need remains in the sector to provide a method for air purification which is adaptable to an indoor and/or domestic environment and has low maintenance costs and a prolonged efficiency over time. The present invention solves the problems of the known art by providing a method for the purification of an indoor and/or domestic environment and simultaneous reduction of CO₂ which, thanks to the maintenance of the algal culture in constant growth conditions, allows to obtain optimized and lasting performance over time.

### SUMMARY OF THE INVENTION

The present invention relates to a method for purifying the air of an indoor and/or domestic environment which exploits a photosynthesis process of an algal culture comprised within an aqueous medium. The method according to the present invention therefore allows to purify the air of an indoor and/or domestic environment by means of the abatement/solubilization of pollutants in the aqueous medium and photo-conversion of CO₂ into O₂ thanks to the photosynthesis process of the algal culture comprised in said aqueous medium. The method according to the present invention further comprises the steps of illuminating and aerating said aqueous medium as well as a step of periodically maintaining said algal culture in a state of constant growth in order to optimize the photosynthesis thereof.

### BRIEF DESCRIPTION OF THE DRAWINGS

FIG. 1 is a first exploded perspective view of a device 1 in accordance with a possible embodiment of the present invention;

FIG. 2 is a section view of the device of FIG. 1;

FIG. 3 is an exploded prospective view of the device of FIG. 1.

FIG. 4 shows the trend of CO₂ subtraction (in mg/m³) in a sealed chamber with the device according to the present invention (a) and without the device according to the present invention (b) as described in Example 2. The dashed vertical line indicates the time when the light source of the device according to the present invention was turned on, and the line (c) indicates the CO₂ concentration present outside the theca.

FIG. 5 shows the trend of O₂ production (in L/m³) in a sealed chamber with the device according to the present invention (a) and without the device according to the present invention (b) as described in Example 2. The dashed vertical line indicates the time at which the light source of the device according to the present invention was turned on.

FIG. 6 shows the trend in the reduction of mould, psychrophilic bacteria, and mesophilic bacteria after 1 hour and 3 hours of operation of the device according to the present invention as described in Example 2.

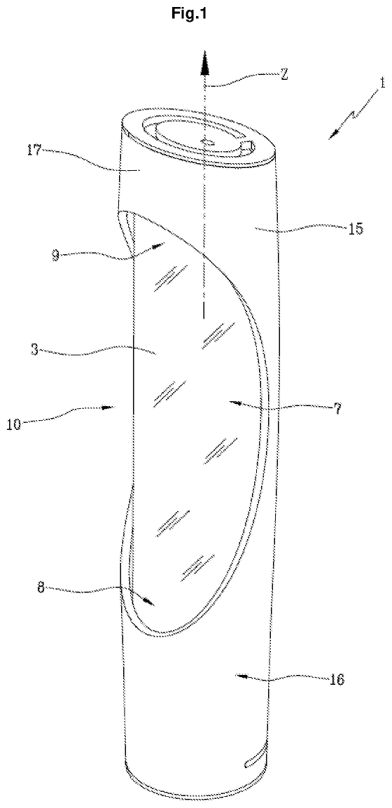

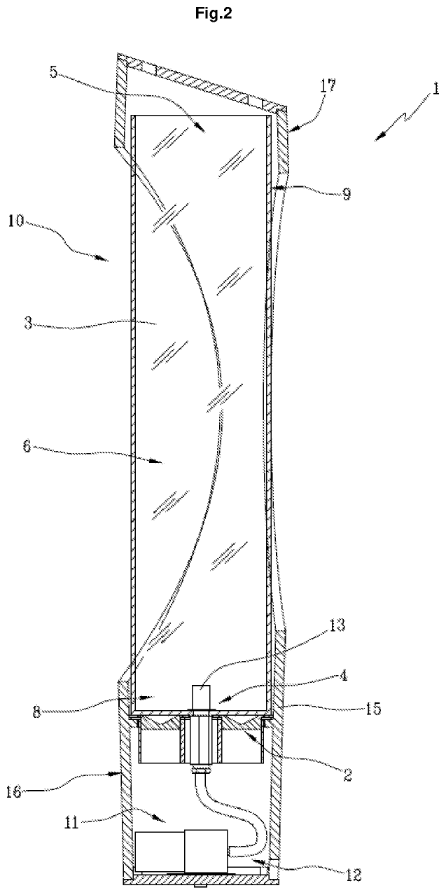

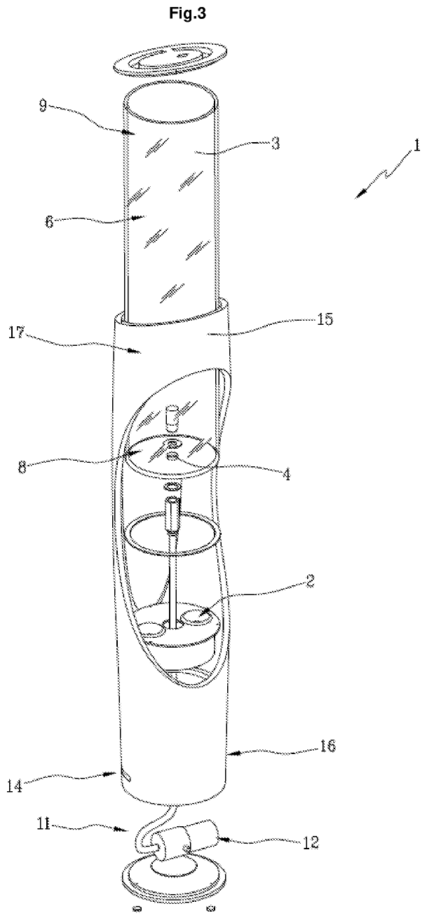

### DETAILED DESCRIPTION OF THE INVENTION

For the purposes of the present invention, “indoor environment” means an enclosed environment, possibly without ventilation, such as a room of an apartment or a house (i.e., a domestic environment), an office, a gym.

The present invention relates to a method for purifying the air of an indoor environment comprising the steps of:
i) providing an aqueous medium comprising an algal culture;
ii) illuminating said aqueous medium with a light source;
iii) conveying an air flow into said aqueous medium, thereby generating a plurality of air bubbles;
said method comprising a step of maintaining said algal culture in a state of constant growth by:
iv) replacing, with a periodic frequency, at least 60%, preferably at least 80%, of the volume of said aqueous medium comprising said algal culture with a corresponding volume of fresh aqueous medium.

Without wishing to be bound by a specific theory, the Applicant has found that the method according to the present invention allows to purify the air of an indoor environment not only by means of the abatement of pollutants possibly present in said air but also by means of the photo-conversion of the carbon dioxide present in said oxygen air. In fact, the method according to the present invention allows to retain and reduce and/or solubilize pollutants possibly present within the air flow conveyed in the aqueous medium. For the purposes of the present invention, said pollutants include carbon dioxide and possibly additional pollutants preferably selected from the group consisting of: particulates and/or fine powders, preferably PM10, PM5 or PM 2,5, or harmful gases such as formaldehyde or nitrogen dioxide moulds, bacteria, preferably psychrophilic bacteria and/or mesophilic bacteria, or a combination thereof. More preferably said further contaminants are selected from: moulds, bacteria, preferably psychrophilic bacteria and/or mesophilic bacteria, or a combination thereof.

Without wishing to be bound to a specific theory, the Applicant has found that the method according to the present invention makes it possible to purify the air of an indoor environment not only by the abatement of said further pollutants possibly present in said air, preferably said moulds and/or bacteria as described above, but also by the photo-conversion of carbon dioxide present in said air to oxygen. The method according to the present invention, in fact, makes it possible to retain and abate and/or solubilize said further polluting agents possibly present within the air flow conveyed in the aqueous medium.

Without wishing to be bound by a specific theory, the Applicant has found that thanks to step iii) of conveying an air flow into said aqueous medium, thereby generating a plurality of air bubbles, it is possible to optimize the exchange surface between the air (comprising the aforementioned pollutants) and the aqueous medium, thus resulting in a more effective abatement and/or solubilization.

The method according to the present invention also allows the photo-conversion of the carbon dioxide present in said oxygen air following the photosynthetic process performed by the algal culture which effectively sequesters the aforesaid pollutants (also including CO₂), producing oxygen and biomass.

For the purposes of the present invention, the term “purifying the air of an indoor environment” therefore means not only a purification from pollutants such as particulates, fine powders, phosphates, formaldehyde or nitrogen dioxide but also a simultaneous production of O₂ from CO₂, resulting in the effective reduction of the concentration of the latter in an indoor environment, even in the absence of ventilation of said environment.

According to a preferred embodiment of the invention, said algal culture comprises or consists of microorganisms selected in the group consisting of: microalgae of the genus Chlorella, cyanobacteria of the genus Arthrospira, green algae, red algae, brown algae, or a combination thereof.

Preferably, said microalgae of the genus Chlorella are selected from the species C. vulgaris, S. sorokiniana, C. pyrenoidosa, or a combination thereof.

Preferably, said cyanobacteria of the genus Arthrospira are for example cyanobacteria belonging to the species A. platensis, so-called “Spirulina”.

In the embodiment in which said algal culture comprises or consists of a combination of said microorganisms, said algal culture is a co-culture, i.e., an algal culture in which said microorganisms are not individually cultured but are co-cultured.

Without wishing to be bound by a specific theory, the Applicant has found that, thanks to steps i)-iv) of the method according to the present invention, the algal culture is configured as a semi-continuous system, i.e., a system in which the algal culture is maintained in a state of constant growth, in particular thanks to the combination of steps ii) and iii) of illumination and aeration with the specific periodic substitution, described in step iv). Said state of constant growth envisages that once the microorganisms have passed from the latency phase (i.e., the period used by the microorganisms to adapt to the environment, i.e., in the case of the present invention, to the aqueous medium) to the exponential growth phase (where the microorganisms multiply rapidly, making the most of the resources present in the aqueous culture medium) to the growth decline phase (where the multiplication rate of the microorganisms gradually begins to decrease), a substantial part of the algal culture is eliminated and replaced with fresh aqueous medium in order to prevent the microorganisms from entering the subsequent stationary phase and decline phase, instead restarting the cycle from the initial latency phase.

Advantageously, step iv) of the method according to the present invention can be considered a maintenance phase which allows to keep the algal culture in the optimal state of growth, ensuring maximum effectiveness in terms of air purification and photosynthetic process for the transformation of CO₂ into oxygen.

Preferably, said step iv) is carried out by replacing, with a periodic frequency, at least 80%, preferably at least 90%, more preferably between 85 and 95% of the volume of said aqueous medium comprising said algal culture with a corresponding volume of fresh aqueous medium.

Preferably, said step iv) is carried out by replacing, with a periodic frequency, between 60 and 90%, more preferably between 60 and 80% of the volume of said aqueous medium comprising said algal culture with a corresponding volume of fresh aqueous medium

For the purposes of the present invention, “fresh aqueous medium” means an aqueous medium, preferably water, which does not comprise an algal culture.

According to a particularly preferred embodiment, said step iv) is carried out with a periodic frequency between once every two weeks and once every 8 weeks, preferably once every three weeks and once every 6 weeks.

Preferably, said periodic frequency is chosen based on the temperature conditions of the environment in which the aqueous medium comprising said algal culture is located; said temperature conditions vary according to the various seasons of the year.

Preferably said environment is an indoor environment, preferably a domestic or work environment such as a room of a house or an apartment, a gym, an office.

According to an embodiment, said step iv) is carried out with a periodic frequency between once every 2 weeks and once every 3 weeks if the temperature of said environment is comprised between 25 and 40° C., preferably between 25 and 35° C.

According to an embodiment, said step iv) is carried out with a periodic frequency between once every 8 weeks and once every 6 weeks if the temperature of said environment is comprised between 5 and 25° C., preferably between 10 and 20° C.

According to an embodiment of the present invention, maintaining said algal culture in a state of constant growth is carried out by maintaining the aqueous medium comprising said algal culture at an average temperature comprised between 15 and 27° C., preferably between 18 and 24° C.

Preferably, the maintenance of said algal culture in a state of constant growth is carried out by maintaining the aqueous medium comprising said algal culture at a homogeneous average temperature, i.e., said aqueous medium having a homogeneous temperature distribution.

Preferably, the maintenance of said aqueous medium at said average temperature, preferably homogeneous in said aqueous medium, is carried out by means of thermal dissipation of the excess heat possibly produced by the light source used in step ii) of the method according to the present invention.

According to a preferred embodiment, the plurality of air bubbles generated by conveying an air flow into the aqueous medium comprising the algal culture according to step iii) is characterized in that said air bubbles have an average diameter of less than 20 mm, preferably less than 5 mm, more preferably comprised between 0.5 mm and 5 mm.

Preferably, said air flow is conveyed into said aqueous medium comprising said algal culture with a flow rate comprised between 5 L/h and 18000 L/h for a volume of aqueous medium comprised between 0.5 and 300 L, preferably with a flow rate comprised between 500 L/h and 1500 L/h for a volume of aqueous medium comprised between 2 and 25 L, more preferably with a flow rate between 50 L/h and 1000 L/h for a volume of aqueous medium between 1.5 and 25 L.

Advantageously, the aforesaid aeration conditions (in particular, the preferred size of the air bubbles and the air flow rate) allow to increase the interface surface between the air flow and the algal culture, thereby increasing the photosynthesis efficiency and, therefore, air purification efficiency according to the method of the present invention.

According to a particularly preferred embodiment, step ii) of illuminating said aqueous medium with a light source and/or said step iii) of conveying an air flow into said aqueous medium, thereby generating a plurality of air bubbles, is carried out for a period of time comprised between 12 and 22 hours, preferably between 14 and 20 hours, more preferably said period of time being a continuous period of time. Advantageously, said lighting conditions create a “day-night” alternation which is particularly preferred for the purposes of the present invention. Advantageously, said aeration conditions create convective motions inside the aqueous medium which allow the algal culture to be homogeneously distributed, keeping it suspended inside said aqueous medium and avoiding an accumulation or sediment which would affect the development and growth conditions of the algal culture itself.

Preferably, said light source is selected from among: sunlight, full-spectrum light, artificial light, preferably an LED, more preferably a plurality of LEDs, or a combination thereof. Preferably said light source is selected from among a white, yellow, red, blue light source or a combination thereof. According to a particular embodiment, the optimal lighting conditions are achieved when a plurality of LEDs are used, preferably said plurality of LEDs having a total luminous flux comprised between 250 and 50000 lumens, preferably 10000 lumens. According to a particularly preferred embodiment, said light source is an LED source, preferably a plurality of LEDs, more preferably having a red light:blue light ratio between 1:1 and 4:1, preferably 3:1.

According to a preferred embodiment, said light source, preferably said plurality of LEDs, creates a total light radiation density within the aqueous medium comprised between 1000 lumens/m 2 and 10000 lumens/m², preferably between 2500 and 8000 lumens/m².

Without wishing to be bound by a specific theory, the Applicant has found that the steps of the method according to the present invention allow to optimize the growth and quality of the state of life of the algal culture, always keeping it in optimal conditions of constant growth, thus resulting in photo-conversion processes deriving from the photosynthesis of said algal culture which are also optimal.

In particular, the preferred “day-night” alternation conditions and the aforementioned aeration conditions, together with the temperature conditions described above and the step iv) of periodic maintenance of the algal culture, effectively allow to maintain said algal culture in the best possible conditions of constant growth and are therefore advantageous for the purposes of the present invention, allowing to maximize the photosynthesis of the algal culture.

Without wishing to be bound by a specific theory, the Applicant has advantageously found that the method according to the present invention allows to accelerate the reduction of the concentration of CO₂ in an enclosed environment to 4-5 hours even if said environment is not ventilated.

The Applicant has also found that the method according to the present invention allows to convert and then eliminate, for each litre of aqueous medium comprising the algal culture, from 10 mg to 240 mg of CO₂ per hour, for a total of 90 g-2.1 kg of CO₂ per year.

According to an embodiment of the present invention, said aqueous medium comprising said algal culture is comprised within a container, preferably a tank, made at least partially of a transparent or translucent material, said material preferably being glass, borosilicate glass, or polymethylmethacrylate.

Preferably, said container has a volume comprised between 0.5 and 300 L, preferably between 2 and 200 L.

According to an embodiment of the present invention, step iii) is carried out by aeration means, preferably a pneumatic pump, adapted to introduce an air flow comprising a predetermined amount of air into said aqueous medium and air diffusion means configured to receive said air flow and generate a plurality of bubbles, preferably said diffusion means comprising an airstone or a porous body.

According to one embodiment, the method according to the present invention consists essentially of the steps (i)-(iv) described above.

According to another embodiment, the method according to the present invention consists of the steps (i)-(iv) described above.

According to a particularly preferred embodiment, with reference to the accompanying drawings, the method according to the present invention is obtained by a device 1 comprising a light source 2 configured to generate a light signal. Furthermore, the device 1 comprises a diffuser body 3 coupled to the light source and configured to propagate the light signal.

In other words, the diffuser body 3 allows the diffusion of the light signal generated by the light source 2.

Preferably, the light source 2 is arranged facing a base portion of the diffuser body 3.

In accordance with a possible embodiment and as illustrated in the accompanying drawings, the light source 2 is arranged below the diffuser body 3 along a direction, preferably vertical, transverse to a support plane of the lighting device 1.

Preferably, moreover, the light source 2 can be arranged below the diffuser body 3 along a vertical direction.

Advantageously, the light source 2 can comprise a plurality of LEDs ensuring the lighting device 1, as well as a longer operating life, a strong reduction in the power absorbed with the same luminous flux generated with respect to the other light sources which are part of the known art.

In particular, the lighting device 1 can comprise a support base adapted to support the aforementioned plurality of LEDs so that it is arranged facing the base portion of the diffuser body 3.

Preferably, the diffuser body 3 is at least partially made of transparent or translucent material, for example glass, borosilicate glass or polymethylmethacrylate or the like, so as to allow an effective diffusion of the light signal. In accordance with a possible embodiment and as illustrated in the accompanying drawings, the diffuser body 3 has a tubular shape, extending along an extension axis “Z”.

According to a purely descriptive and non-limiting embodiment of the present invention, the diffuser body 3 can have a base diameter comprised between 30 and 1000 mm, preferably 100, and a height comprised between 50 and 2000 mm, preferably 370.

Preferably, during a configuration of use of the lighting device 1, the extension axis “Z” is substantially parallel to a vertical direction so that the diffuser body 3 can ensure a broad diffusion of the light signal. According to further possible embodiments not illustrated in the accompanying drawings, the diffuser body can have a different shape, for example cubic, parallelepiped or substantially spherical, without altering the inventive concept underlying the present invention.

The diffuser body 3 further has at least one inlet opening 4 and at least one outlet opening 5 operatively connected to allow the passage of an air flow.

In other words, the diffuser body 3 can define a containment volume 6 adapted to allow the transit of the aforementioned air flow.

Advantageously, the openings 4, 5 allow the circulation of an air flow so that it is purified by special air purification means 7, as will be described in detail below.

In accordance with a possible embodiment and as illustrated in the accompanying drawings, the diffuser body 3 can have a tubular shape having a first end 8 and a second end 9. In particular, at least one inlet opening 4 can be arranged at the first end 8 of the diffuser body 3 and at least one outlet opening 5 can be arranged at a second end 9 of the diffuser body 3 opposite the first end 8 and/or at a side surface of the same diffuser body 3.

The device 1 further comprises an air purification means 7 arranged at least partially inside the diffuser body 3 and configured to remove pollutants present in the air flow entering the diffuser body 3.

Advantageously, the air purification means 7 can comprise a photo-bioreactor 10 comprising an algal culture adapted to photo-convert the carbon dioxide to oxygen and/or to purify the air flow by means of a photosynthesis process of said algal culture, thus advantageously allowing the implementation of the method according to the present invention.

In particular, the diffuser body 3 can be configured to house the aforementioned algal culture inside the containment volume 6.

In other words, the photo-bioreactor 10 is configured to purify the air flow entering the diffuser body 3 through at least one inlet opening 4. The air flow interacts with the algal culture housed inside the containment volume 10 allowing the photosynthesis thereof and is subsequently emitted, substantially free of pollutants, through the at least one outlet opening 5.

The photosynthesis also contributes to the development of the algal culture, which, with a frequency depending on the growth rate of the same culture, must be at least partially removed from the containment volume 6 to allow the continuation of the air purification.

Advantageously, said partial removal from the containment volume is equivalent to a periodic maintenance of the algal culture, to which it allows the effective maintenance of the algal culture in its continuous growth phase, thus allowing a virtually unlimited life of the culture itself.

In accordance with a possible non-limiting embodiment of the present invention, the algal culture can comprise or consist of microalgae of the genus Chlorella, including but not limited to the species C. vulgaris, C. sorokiniana, and C. pyrenoidosa.

According to further possible embodiments of the present invention, the aforementioned algal culture can comprise or consist of cyanobacteria of the genus Arthrospira, including but not limited to the species A. platensis, so-called “Spirulina”. Likewise, further embodiments of the present invention can comprise other types of photosynthetic microorganisms, including but not limited to cyanobacteria, green algae, red algae, and brown algae, both individually grown and co-cultured, without altering the inventive concept underlying the present invention.

Preferably, said algal culture is as previously described.

In other words, therefore, the air purification means 7 may advantageously comprise a photobioreactor 10 comprising an algal culture or co-culture suitable for photo-converting carbon dioxide to oxygen and/or purifying the air flow by a photosynthesis process of said algal culture or co-culture.

In particular, the diffuser body 3 may be configured to house the aforementioned algal culture or co-culture within the containment volume 6.

In other words, the photobioreactor 10 is configured to purify the air flow entering the diffuser body 3 through at least one inlet opening 4. The air flow interacts with the algal culture or co-culture housed within the containment volume 10 allowing it to photosynthesize and is subsequently emitted, with a pollutant content reduced relative to the content of the incoming air flow (preferably substantially pollutant-free), through the at least one outlet opening 5.

In other words, the photobioreactor 10 allows the lighting device 1 to produce oxygen.

Advantageously, the inlet opening 4 obtained at the base portion of the diffuser body 3 facilitates the diffusion of the air flow into the algal culture, promoting an effective development of the photosynthesis reactions.

The device 1 can further comprise aeration means 11 adapted to introduce a predetermined amount of air inside the diffuser body 3 through the at least one inlet opening 4.

Advantageously, the aeration means 11 can comprise a pneumatic pump 12 adapted to promote the inflow of air to the air purification means 7.

Thereby, the device 1 promotes the entry of the air flow into the containment volume 6 ensuring reduced dimensions and limited noise pollution with respect to the devices of the prior art.

Furthermore, the device 1 can comprise an air diffusion means 13 facing the inlet opening 4.

The air diffusion means 13 is configured to receive the air flow passing through the inlet opening 4 and generate a plurality of bubbles having an average diameter of less than 20 mm, preferably less than 5 mm, more preferably comprised between 0.5 mm and 5 mm.

In particular, the air diffusion means 13 can comprise an airstone or a porous body and, being arranged at the inlet opening 4, receive the air flow coming, preferably, from the aeration means 11 and break it down into a plurality of bubbles.

Thereby, the diffusion means allows to increase the interface surface between the air flow and the algal culture, thereby increasing the photosynthesis efficiency and, therefore, the air purification efficiency according to the method of the present invention.

Advantageously, the device 1 can further comprise a heat dissipation means 14 configured to dissipate an amount of heat generated by the light source 2 so as to ensure an algal development in accordance with specific temperature conditions and allow the desired constant growth conditions according to the method of the present invention.

Preferably, the heat dissipation means 14 comprises one or more of at least one ventilation opening, adapted to allow an effective inflow of air towards the light source 2, and/or a fan, configured to promote a removal of an amount of air at high temperature from the vicinity of the light source 2.

Advantageously, the lighting device 1 can further comprise a control and adjustment module configured to allow the modification of one or more operating parameters of the lighting device 1 itself.

In accordance with a purely illustrative and non-limiting embodiment of the present invention, the control and adjustment module can allow the adjustment of the intensity and/or wavelength of the light signal and/or can allow the adjustment of the air flow entering the diffuser body 3 by adjusting, for example, the aeration means 11. In other words, the control and adjustment module can allow the adjustment of the light signal emitted by the LEDs and the adjustment of the flow rate of the air flow moved by the pneumatic pump 12.

It should be underlined that such adjustments allow an effective performance of the photosynthesis reactions inside the containment volume 6, preferably, adjusting the algal development so as to optimize the removal of the pollutants present in the air flow entering the diffuser body 3.

Preferably, the control and adjustment module can allow the adjustment of the aforementioned operating parameters automatically and/or manually thanks to the intervention of a user.

In particular, the control and adjustment module can comprise wireless interface means, for example of the Bluetooth or Wi-Fi type, adapted to allow a user to carry out the previously described adjustments by means of a remote control and/or an app for mobile devices (for example smartphone or tablet).

In accordance with a possible embodiment and as illustrated in the accompanying drawings, the device 1 can comprise a containment body 15 defining a housing adapted to at least partially house the diffuser body and/or the light source, giving the device 1 particular structural strength and aesthetic pleasantness.

In particular, the containment body 15 can comprise a support portion 16 adapted to house a respective base portion of the diffuser body 3, the light source 2 and the aeration means 11.

Preferably, the support portion 16 defines a support portion adapted to rest on a support platform for the device 1 and can comprise a non-slip portion adapted to increase the stability of the device 1.

The containment body 15 can further comprise a top portion 17.

Preferably, the top portion 17 is fitted to the second end 9 of the diffuser body 3 and/or has at least one opening adapted to allow the passage of air flow from the diffuser body 3 to an environment outside the device 1.

The support portion 16 and the top portion 17 can be made in a single piece, giving the containment body 15 a monolithic structure of high strength.

According to further possible embodiments, the support portion 16 and the top portion 17 can be made separately without altering the inventive concept underlying the present invention.

Advantageously, the containment body 15 can be made of a plurality of different materials, for example wood, metal and/or polymeric materials, ensuring a highly flexible design and high aesthetic pleasantness for the device.

According to an embodiment said device 1 is a lighting device.

It should therefore be noted that the present invention achieves the proposed objects by providing a method for purifying the air of an indoor environment preferably by means of the device as described above. The method according to the present invention advantageously allows to increase the liveability conditions inside an environment thanks to the air purification configured to remove pollutants present in an air flow which passes therethrough.

Advantageously, the lighting device has limited dimensions and low noise which give it high operating flexibility and adaptability to the environments of use.

Advantageously, moreover, the control and adjustment module allows to adjust the temperature of the algal culture and the air inflow to the containment volume and, therefore, the volume of the purified air, thereby allowing an efficient adaptation of the lighting device to the environment in which it is arranged.

### EXAMPLES

### Example 1

The device according to the present invention as described in the claims has been tested to evaluate the CO₂ subtraction capability. More specifically, the device was placed in a 1 m³ sealed chamber inside which the CO₂ concentration was 1000 ppm (equivalent to the CO₂ concentration inside a well-ventilated office). It was then possible to measure a CO₂ subtraction rate of up to 92 mg/h, equivalent to 25 Kenzia plants.

### Example 2

The test as described in Example 1 was repeated by varying the CO₂ concentration inside the theca and inserting additional pollutants in order to simulate extremely polluted environments. The device according to the present invention was placed in a 1 m³ sealed chamber containing air polluted with bacteria (psychrophilic and mesophilic), mould and 10000 ppm CO₂. The light source (i.e., plurality of LEDs) of the device according to the present invention was turned off overnight, and turned on at 7:30 AM.

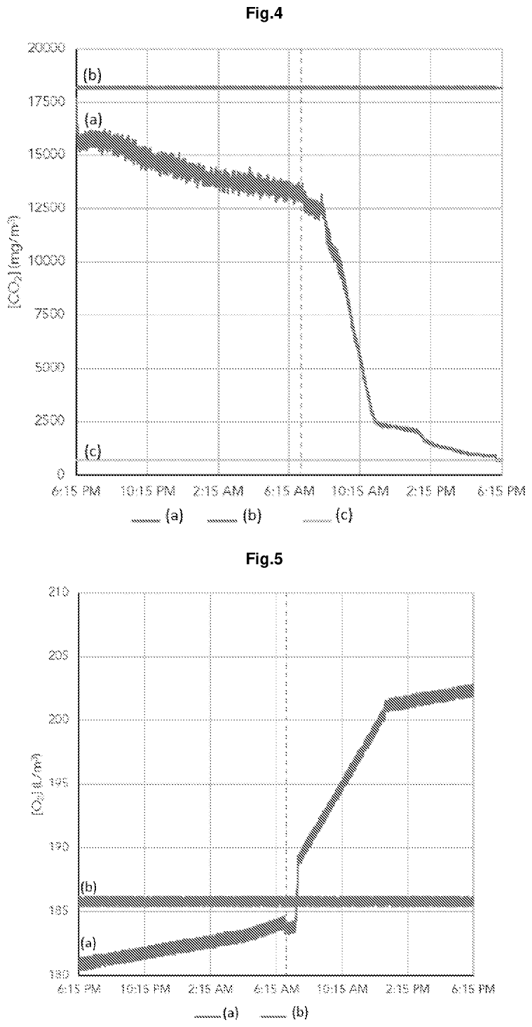

As shown in FIG. 4, within a few hours, the device (operating the method of the present invention) removed 95% of the CO₂, bringing the concentration back to the same levels as measured outside the theca (i.e., 500 ppm). During such an experiment, it was possible to observe a CO₂ sequestration rate of about 1800 mg/h, equivalent to 500 Kenzia plants.

Similarly, as shown in FIG. 5, oxygen production was measured. Over the course of 24 hours, the device according to the present invention increased the oxygen concentration in the theca from 19.6% to 22.1%, or an increase of 13% corresponding to about 22 L of oxygen.

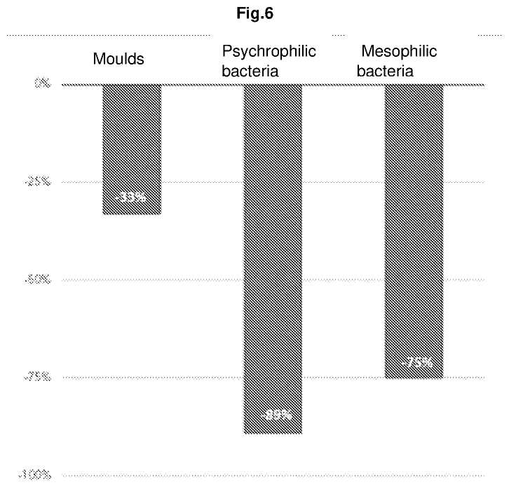

The ability of the device according to the present invention to break down mould and bacteria present within the air in the case was also measured. As shown in FIG. 6, after only one hour after turning on the device, a 33% reduction in mould was measured. Three hours after the device was turned on, an 89% reduction in bacteria of environmental origin (psychrophilic bacteria) and a 75% reduction in bacteria of human and animal origin (mesophilic bacteria) was measured.

1. A method for purifying the air of an indoor environment, comprising the steps of:
i) providing an aqueous medium comprising an algal culture;
ii) illuminating said aqueous medium with a light source;
iii) conveying an air flow into said aqueous medium, thereby generating a plurality of air bubbles having an average diameter of less than 20 mm;
iv) maintaining said algal culture in a state of constant growth by replacing, with a periodic frequency, between 1 time every two weeks and 1 time every 8 weeks, an amount between 60% and 80%, of the volume of said aqueous medium comprising said algal culture with a corresponding volume of fresh aqueous medium and maintaining the aqueous medium comprising said algal culture at an average temperature between 15 and 27° C.

2. The method according to claim 1, wherein said algal culture comprises or consists of microorganisms selected in the group consisting of: microalgae of the genus Chlorella, cyanobacteria of the genus Arthrospira, green algae, red algae, brown algae, or a combination thereof.

3. The method according to claim 1, wherein said step iv) is carried out with a periodic frequency comprised between once every three weeks and once every 6 weeks.

4. The method according to claim 1, wherein maintaining said algal culture in a state of constant growth is achieved by maintaining the aqueous medium comprising said algal culture at an average temperature comprised between 18 and 24° C.

5. The method according to claim 1, wherein said bubbles have an average diameter of less than 5 mm, more preferably comprised between 0.5 mm and 5 mm.

6. The method according to claim 1, wherein said air flow is conveyed into said aqueous medium at a flow rate comprised between 5 L/h and 18000 L/h fora volume of aqueous medium comprised between 0.5 and 300 L, preferably between 500 L/h and 1500 L/h for a volume of aqueous medium comprised between 2 and 25 L, more preferably between 50 L/h and 1000 L/h for a volume of aqueous medium between 1.5 and L.

7. The method according to claim 1, wherein said step ii) of illuminating said aqueous medium with a light source and/or said step iii) of conveying an air flow into said aqueous medium, thereby generating a plurality of air bubbles, are carried out for a period of time comprised between 12 and 22 hours, preferably between 14 and 20 hours, more preferably said period of time being a continuous period of time.

8. The method according to claim 1, wherein said light source is selected from among: sunlight, full-spectrum light, artificial light, preferably an LED, more preferably a plurality of LEDs, or a combination thereof.

9. The method according to claim 1, wherein said aqueous medium comprising said algal culture is comprised inside a container, preferably a tank, made at least partly of a transparent or translucent material, said material being preferably glass, borosilicate glass, or polymethylmethacrylate.

10. The method according to claim 1, wherein said step iii) is carried out using an aeration means, preferably an air pump, adapted to inject an air flow comprising a predetermined amount of air into said aqueous medium and an air diffusion means configured to receive said flow of air and to generate a plurality of bubbles, said diffusion means preferably comprising an airstone or a porous body.
